(19) United States
(12) Patent Application Publication
(10) Pub. No.: US 2007/0260478 A1
(43) Pub. Date: Nov. 8, 2007

(54) Delivery of Health Insurance Plan Options

(75) Inventors: Robert M. Szabo, Boca Raton, FL (US); James J. Toohey, Boca Raton, FL (US)

Correspondence Address: CUENOT & FORSYTHE, L.L.C., 12230 FOREST HILL BLVD., STE. 120, WELLINGTON, FL, 33414, US

(73) Assignee: International Business Machines Corporation, Armonk, NY (US)

(21) Appl. No.: 11/381,136

(22) Filed: May 2, 2006

(51) Int. Cl. G06Q 10/00; A61B 5/00

(52) U.S. Cl. 705/2; 600/300

(57) ABSTRACT

A method for providing approved medical treatment options. The method can include receiving a patient identifier from a first client via a communications network, receiving at least one diagnostic code from the first client via the communications network, and, in real time, automatically selecting at least one treatment identifier that corresponds to the patient identifier and the diagnostic code. The present invention also can include a system for providing approved medical treatment options. The system can include a retrieval application that receives a patient identifier and at least one diagnostic code from a first client via a communications network. The system can, in real time, automatically select at least one treatment identifier that corresponds to the patient identifier and the diagnostic code.

### BACKGROUND

1. Field of the Invention

The present invention relates to the field of data communications and, more particularly, to communication of medical treatment options.

2. Description of the Related Art

In recent years the concept of managed care has evolved into a wide assortment of health insurance options. Managed care plans provide healthcare coverage that is relatively affordable by imposing certain restrictions and guidelines on the consumer. Examples of such plans are those provided by health maintenance organizations (HMOs), preferred provider organizations (PPOs), and point of service (POS) plans.

In an HMO, the consumer chooses a primary care physician to coordinate their medical care and act as a “gatekeeper,” coordinating the consumer's medical care from a list of available participating providers contracted with the particular HMO. Generally speaking, all medical care must be approved by the primary care physician, and visits to specialists are only allowed with a referral from the primary care physician. Unapproved visits to other healthcare providers typically are not covered under an HMO's insurance plan.

Insurance coverage under a PPO is more similar to a traditional indemnity insurance plan in the sense that no primary care physician is required. The PPO plan pays a higher percentage for the use of a “preferred provider” whose name appears on the plan's preferred provider list, however.

In a POS the consumer must select a primary care physician upon enrollment. Should specialized care be necessary, the consumer (i.e. patient) has the option of going through the primary care physician for a referral to a participating POS specialist for the greatest amount of insurance coverage. In the event that the specialist is not a member of the POS, coverage will still be provided, but at a lower rate.

The wide assortment of managed care plans that are currently available complicates patient treatment. When a patient insured under a particular healthcare plan visits his physician, the patient must be able to inform the doctor, for example, what treatment options are covered under the plan. It is therefore generally recommended that the patient provide a list of such treatment options. If a patient fails to do so, he runs the risk of receiving a prescription for medication or a referral that is not covered under the plan.

The lists of treatment options are tedious to compile and their accuracy may vary. Indeed, plan lists are subject to change and the lists are frequently out of date. Oftentimes new lists can be downloaded via the Internet, but gathering such lists requires much forethought and planning and is not conducive to last minute preparations. For example, the patient is typically required to enter a specific plan number, identification information, and the like. Moreover, the lists tend to be rather long and thus take a physician much time to process.

It would be beneficial to provide a service that simplifies accurate identification of treatment options that are covered under a patient's healthcare plan.

### SUMMARY OF THE INVENTION

The present invention provides methods, a system, and apparatus that facilitate identification of medical treatment options that are covered under a patient's healthcare plan. For example, one embodiment of the present invention can include a method for providing approved medical treatment options. The method can include receiving a patient identifier from a first client via a communications network, receiving at least one diagnostic code from the first client via the communications network and, in real time, automatically selecting at least one treatment identifier that corresponds to the patient identifier and the diagnostic code.

Another embodiment of the present invention can include a system for providing approved medical treatment options. The system can include a retrieval application that receives a patient identifier and at least one diagnostic code from a first client via a communications network. The system can, in real time, automatically select at least one treatment identifier that corresponds to the patient identifier and the diagnostic code.

Yet another embodiment of the present invention can include a machine readable storage being programmed to cause a machine to perform the various steps and/or functions described herein.

### BRIEF DESCRIPTION OF THE DRAWINGS

There are shown in the drawings, embodiments which are presently preferred, it being understood, however, that the invention is not limited to the precise arrangements and instrumentalities shown.

FIG. 1 is a block diagram depicting a system that provides treatment product and service provider options in accordance with one embodiment of the present invention.

FIG. 2 is a flow chart illustrating a method of updating approved treatment product and service provider options in accordance with another embodiment of the present invention.

FIG. 3 is a flow chart illustrating a method of retrieving approved treatment product and service provider options in accordance with another embodiment of the present invention.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

The present invention relates to a service that facilitates identification of medical treatment that is covered under a patient's healthcare plan. The service can be, for example, provided by a third party that maintains records of currently available healthcare plan options for each of a plurality of healthcare plans. The healthcare plans can be those plans that subscribe to the service, plans that cover a significant number of people, and/or plans identified by clinicians who subscribe to the service. Thus, a clinician can use the same service to retrieve authorized treatment options for each of his patients, even if the patients use different healthcare plans.

During a patient encounter, a clinician can identify a patient to the service. The clinician also can enter diagnostic codes that correlate to the patient's condition. Based on the patient identification and the diagnostic codes, the service can return a list of treatment products (e.g. pharmaceutical and/or therapeutic products) and/or a list of service providers that are approved under the patient's healthcare plan for treating the patient's condition. In the case that an approved treatment product is a pharmaceutical, approved dosages also can be returned with the lists. From the provided lists the clinician can choose suitable treatment products to prescribe to the patient, dosages (if applicable) and/or service provider(s) to whom to refer the patient. By providing accurate and well targeted lists in this manner, the clinician is able to efficiently select the most appropriate treatment products and/or service providers for the patient.

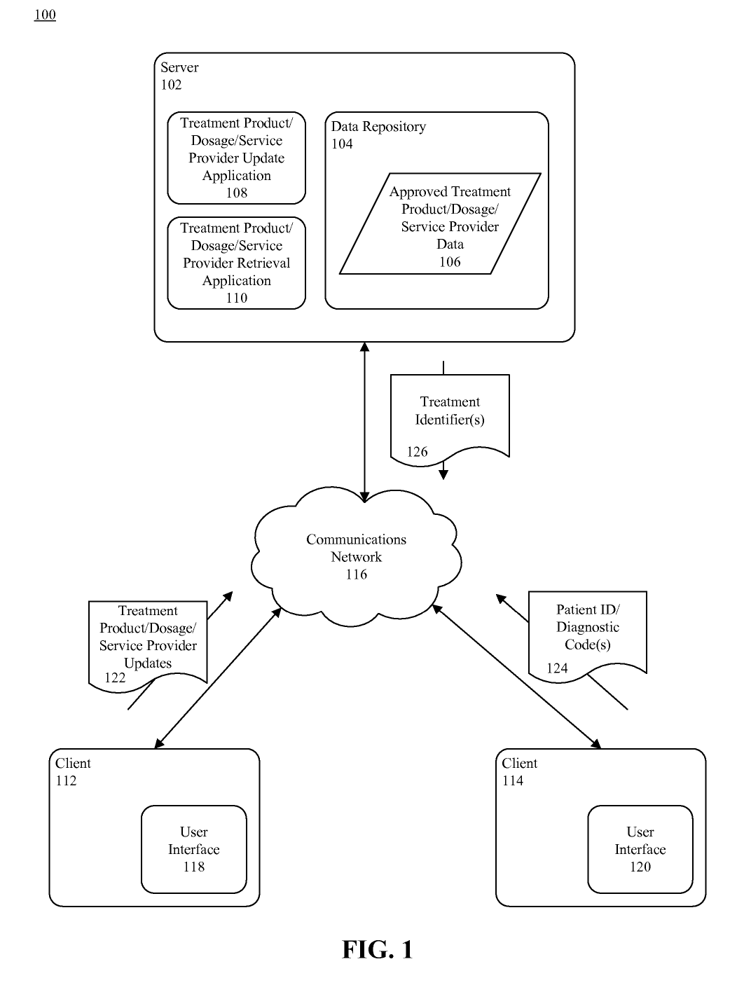

FIG. 1 is a block diagram depicting a system 100 that provides treatment product, dosage and service provider options in accordance with one embodiment of the present invention. The system 100 can include a server 102 operatively coupled to a data repository 104 that contains data 106. The data 106 can include approved treatment products, dosages and service providers for one or more healthcare plans. The data 106 also can include patient identifiers, patient profiles, patient medical histories, service provider profiles, and/or any other desired information.

The data 106 can be stored in the data repository 104 using one or more data files, text files, data tables, or stored in any other suitable manner. For example, the data repository 104 can include a database having one or more data tables in which the data 106 is stored. The data tables can, for instance, include records that associate patient identifiers with healthcare plans, associate diagnostic codes with approved treatment products and, where applicable, dosages, associate diagnostic codes with approved service providers, and associate patient identifiers and service providers with geographic regions. Still, a myriad of other data can be included in the database and the invention is not limited in this regard.

A treatment product/service provider update application (hereinafter “update application”) 108 and a treatment product/service provider retrieval application (hereinafter “retrieval application”) 110 can be provided to process the data 106. The update and retrieval applications 108, 110 can be configured for use by clients 112, 114 that communicate with the server 102 via a communications network 116. For example, the update and retrieval applications 108, 110 can be network based applications. In that regard, the communications network 116 can be the World Wide Web, the Internet, an intranet, a wide area network (WAN), a local area network (LAN), a cellular communications network, or any other communications network suitable for communicating digital data. As such, the communications network 116 can include wired and/or wireless communication links.

The clients 112, 114 each can include a respective user interface 118, 120. In one arrangement the user interfaces 118, 120 can be graphical user interfaces, for instance web browsers. Nonetheless, the invention is not so limited and any other suitable types of user interfaces can be used. For example, either or both of the user interfaces 118, 120 can be applications specifically developed to implement the methods described herein. Further, either or both of the user interfaces 118, 120 can be speech based. In such an arrangement the user interfaces 118, 120 can include speech recognition and/or text-to-speech synthesis. Speech recognition and text-to-speech synthesis are known to the skilled artisan.

In operation the user interface 118 on the client 112 can be used by a healthcare plan administrator to communicate treatment product and/or service provider updates 122 to the update application 108. The update application 108 can update the data 106 with the treatment product and/or service provider updates 122 in real time. As used herein, the term “real time” means a level of processing responsiveness that a user senses as sufficiently immediate or that enables the processor to keep up with some external process.

The user interface 120 on the client 114 can be used by a clinician to communicate patient identifiers and diagnostic codes 124 to the retrieval application 110. The retrieval application 110 can process the patient identifiers and diagnostic codes 124 to select suitable treatment identifiers 126 from data the 106. For example, the retrieval application 110 can process the patient identifier to identify a patient's healthcare provider. The retrieval application 110 also can process the diagnostic code(s) to identify treatment products, dosages, and/or service providers that are approved under the patient's healthcare plan to treat the patient's condition. The service providers identified in the list can be limited to those within a particular geographic region. The geographic region can be a region identified by the clinician or a region that has been identified in the patient's profile. The treatment identifier(s) 126 then can be communicated to the client 114 and presented to the clinician via the user interface 120. For instance, the treatment identifier(s) can be presented in one or more lists. The clinician then can peruse the lists to select one or more treatment products to prescribe to the patient and/or one or more service providers to which to refer the patient.

Notably, because the data 106 is updated in real time, the treatment identifiers 126 retrieved by the clinician will be current. Accordingly, the risk of prescribing medications or issuing referrals to service providers that are no longer approved is minimized. Moreover, because the treatment identifiers 126 are selected based on a patient identifier and diagnostic codes entered by the clinician, the list of treatment products, dosages and/or service providers that is presented to the clinician can be limited to those that are both approved by the patient's healthcare plan and are suitable for treating the patient's condition. With a list targeted in this manner, the clinician can quickly and efficiently choose desired pharmaceutical products to prescribe to the patient and/or service providers to whom to refer the patient.

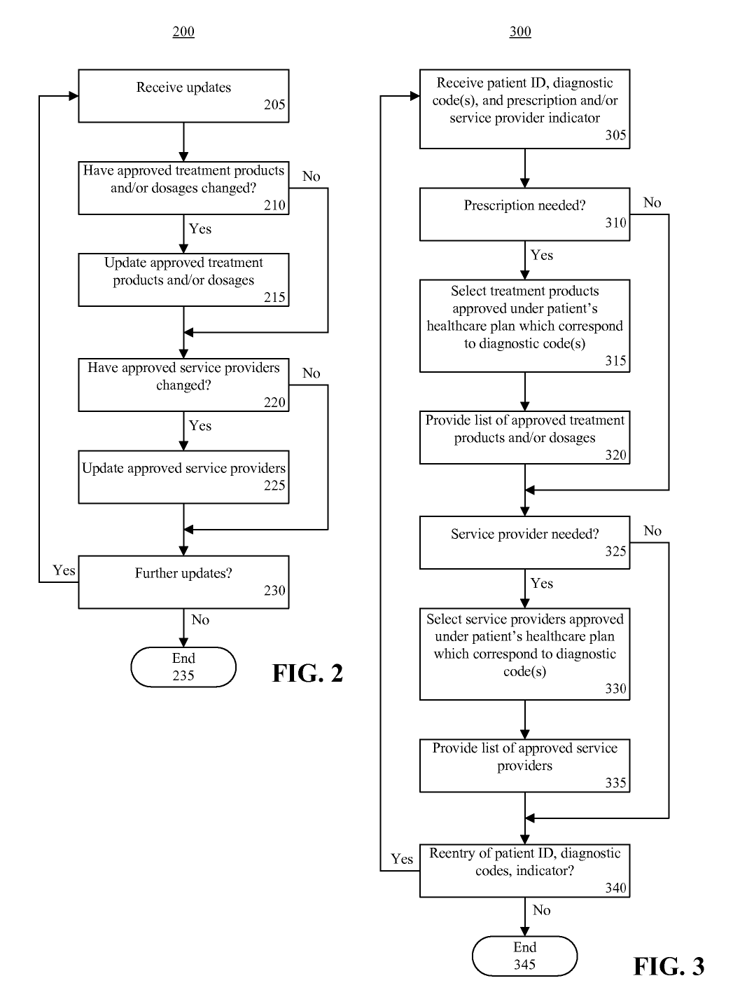

FIG. 2 is a flow chart illustrating a method 200 of updating approved treatment product and service provider options in accordance with another embodiment of the present invention. The method 200 can begin in a state in which a user of a client, for example a healthcare plan administrator, has been authenticated and a user session has been established between a client and a server. At step 205 an update application instantiated on the server can receive treatment product, dosage and/or service provider updates from the client. The updates can be automatically associated with a healthcare plan for which the user is approved to enter updates.

Referring to decision box 210 and step 215, if the update contains a treatment product update, the update application can update the list of treatment products and dosages (if applicable) that are approved by the healthcare plan. For example, treatment products can be added, modified or deleted from a list of approved treatment products. Such additions, modifications or deletions can be performed in real time.

Referring to decision box 220 and step 225, if the update contains a service provider update, the update application can update the list of service providers that are approved by the healthcare plan. For example, service providers can be added, modified or deleted from a list of approved service providers. Such additions, modifications or deletions also can be performed in real time.

Referring to decision box 230, if further updates are requested, the process can repeat. If, however, all requested updates are complete, the process can end at step 235.

FIG. 3 is a flow chart illustrating a method 300 of retrieving approved treatment product and service provider options in accordance with another embodiment of the present invention. The method 300 can begin in a state in which a user of a client, for example a clinician, has been authenticated and a user session has been established between the client and the server. At step 305 a retrieval application instantiated on the server can receive from the client a patient identifier and one or more diagnostic codes. An indicator also can be received indicating whether a prescription for a treatment product is needed and whether a referral to an approved service provider is needed.

Referring to decision box 310 and step 315, if a prescription for a treatment product is needed, the retrieval application can process the patient identifier to identify the patient's healthcare plan and select treatment products approved under the patient's healthcare plan which correspond to the diagnostic code(s). If any of the selected treatment products are pharmaceuticals, approved dosages also can be identified. At step 320 a list of the selected treatment products and dosages (if applicable) can be provided to the client and presented to the user.

Referring to decision box 325 and step 330, if a referral to a service provider is needed, the retrieval application can process the patient identifier to identify the patient's healthcare plan and select service providers approved under the patient's healthcare plan which correspond to the diagnostic code(s). At step 335 a list of the service providers can be provided to the client and presented to the user. In one arrangement, if both a prescription and a referral are needed, the list of approved treatment products and the list of approved service providers can be simultaneously presented.

Referring to decision box 340, if the clinician enters a new patient identifier, diagnostic code(s) or prescription/referral indicator, the process can repeat. For example, the clinician can correct such information if the information was entered incorrectly or if the clinician wishes to change his diagnosis. Otherwise, the process can end at step 345.

The present invention can be realized in hardware, software, or a combination of hardware and software. The present invention can be realized in a centralized fashion in one computer system or in a distributed fashion where different elements are spread across several interconnected computer systems. Any kind of computer system or other apparatus adapted for carrying out the methods described herein is suited. A typical combination of hardware and software can be a general-purpose computer system with a computer program that, when being loaded and executed, controls the computer system such that it carries out the methods described herein. The present invention also can be embedded in a computer program product, which comprises all the features enabling the implementation of the methods described herein, and which when loaded in a computer system is able to carry out these methods.

The terms “computer program”, “software”, “application”, variants and/or combinations thereof, in the present context, mean any expression, in any language, code or notation, of a set of instructions intended to cause a system having an information processing capability to perform a particular function either directly or after either or both of the following: a) conversion to another language, code or notation; b) reproduction in a different material form. For example, a computer program can include, but is not limited to, a subroutine, a function, a procedure, an object method, an object implementation, an executable application, an applet, a servlet, a source code, an object code, a shared library/dynamic load library and/or other sequence of instructions designed for execution on a computer system.

The terms “a” and “an”, as used herein, are defined as one or more than one. The term “plurality”, as used herein, is defined as two or more than two. The term “another”, as used herein, is defined as at least a second or more. The terms “including” and/or “having”, as used herein, are defined as comprising, i.e. open language. The term “coupled”, as used herein, is defined as connected, although not necessarily directly, and not necessarily mechanically, i.e. communicatively linked through a communication channel or pathway or another component or system. The term “service provider”, as used herein, is any entity that may provide services that are covered under a healthcare plan. For example, a service provider can be a hospital, lab, physician, therapist, etc. The term “treatment product”, as used herein, is any product that can be used to treat a patient. For example, a treatment product can be a medication, an appliance, a bandage, or any other product that can be used to treat a patient's condition. As used herein, the term “medical treatment” means any service and/or product that can be used to treat a patient.

This invention can be embodied in other forms without departing from the spirit or essential attributes thereof Accordingly, reference should be made to the following claims, rather than to the foregoing specification, as indicating the scope of the invention.

What is claimed is:

1. A method for providing approved medical treatment options comprising:
receiving a patient identifier from a first client via a communications network;
receiving at least one diagnostic code from the first client via the communications network; and
in real time, automatically selecting at least one treatment identifier that corresponds to the patient identifier and the diagnostic code.

2. The method of claim 1, further comprising:
determining a particular healthcare plan associated with the patient identifier;
wherein selecting the treatment identifier comprises selecting the treatment identifier from a plurality of treatment identifiers that are approved under the healthcare plan.

3. The method of claim 1, wherein selecting the treatment identifier comprises selecting the treatment identifier from the group consisting of an approved treatment product, an approved dosage and an approved service provider.

4. The method of claim 1, further comprising communicating the treatment identifier to the first client via the communications network.

5. The method of claim 1, further comprising:
receiving treatment identifier updates from a second client; and
in real time, updating at least one approved treatment identifier list.

6. The method of claim 5, wherein receiving the treatment identifier updates comprises receiving the treatment identifier updates via the communications network.

7. The method of claim 5, wherein receiving the treatment identifier updates comprises receiving at least one treatment identifier selected from the group consisting of an approved treatment product, an approved dosage and an approved service provider.

8. A system for providing approved medical treatment options comprising a retrieval application that receives a patient identifier and at least one diagnostic code from a first client via a communications network and, in real time, automatically selects at least one treatment identifier that corresponds to the patient identifier and the diagnostic code.

9. The system of claim 8, wherein the retrieval application determines a particular healthcare plan associated with the patient identifier and selects the treatment identifier from a plurality of treatment identifiers that are approved under the healthcare plan.

10. The system of claim 8, wherein the treatment identifier is selected from the group consisting of an approved treatment product, an approved dosage and an approved service provider.

11. The system of claim 8, wherein the retrieval application communicates the treatment identifier to the first client via the communications network.

12. The system of claim 8, further comprising an update application that receives treatment identifier updates from a second client and, in real time, updates at least one approved treatment identifier list.

13. The system of claim 12, wherein the treatment identifier updates are received via the communications network.

14. The system of claim 12, wherein the treatment identifier is selected from the group consisting of an approved treatment product, an approved dosage and an approved service provider.

15. A machine readable storage, having stored thereon a computer program having a plurality of code sections comprising:
code for receiving a patient identifier from a first client via a communications network;
code for receiving at least one diagnostic code from the first client via the communications network; and
code for, in real time, automatically selecting at least one treatment identifier that corresponds to the patient identifier and the diagnostic code.

16. The machine readable storage of claim 15, further comprising:
code for determining a particular healthcare plan associated with the patient identifier;
wherein the code for selecting the treatment identifier comprises code for selecting the treatment identifier from a plurality of treatment identifiers that are approved under the healthcare plan.

17. The machine readable storage of claim 15, wherein the code for selecting the treatment identifier comprises code for selecting the treatment identifier from the group consisting of an approved treatment product, an approved dosage and an approved service provider.

18. The machine readable storage of claim 15, further comprising code for communicating the treatment identifier to the first client via the communications network.

19. The machine readable storage of claim 15, further comprising:
code for receiving treatment identifier updates from a second client; and
code for, in real time, updating at least one approved treatment identifier list.

20. The machine readable storage of claim 19, wherein the code for receiving the treatment identifier updates comprises code for receiving at least one treatment identifier selected from the group consisting of an approved treatment product, an approved dosage and an approved service provider.
